(19) United States
(12) Patent Application Publication
(10) Pub. No.: US 2001/0047949 A1
(43) Pub. Date: Dec. 6, 2001

(54) Cosmetic applicator with transparent tip

(76) Inventor: Doreen Bono, Dix Hills, NY (US)

Correspondence Address: James M. Loeffler, Esq., Dilworth & Barrese, LLP, 333 Earle Ovington Boulevard, Uniondale, NY, 11553, US

(21) Appl. No.: 09/794,845

(22) Filed: Feb. 27, 2001

Related U.S. Application Data
(60) Provisional application No. 60/186,942, filed on Mar. 4, 2000.

(51) Int. Cl. A61L 15/00

(52) U.S. Cl. 206/438

(57) ABSTRACT

A cosmetic applicator for applying substantially solid cosmetic material comprises a container defining at least one cavity to receive the cosmetic material and including an aperture disposed adjacent a distal end portion thereof in communication with the at least one cavity, the container further including a transparent portion to determine the amount of the cosmetic material remaining within the cosmetic applicator. The cosmetic applicator may further include a cosmetic moving member for extracting and retracting the cosmetic material from and/or within the aperture.

### CROSS-REFERNCE TO RELATED APPLICATIONS

This application claims the benefit of U.S. Provisional Application No. 60/186,942 filed on Mar. 4, 2000.

### BACKGROUND

This version of the invention is concerned with the field of cosmetic applicators. More specifically, this version of the invention is concerned with cosmetic applicators that are constructed with a clear or transparent tip or end portion that permits the user to determine the amount of cosmetic remaining within the applicator.

### PRIOR ART

Cosmetics or makeup that are applied to the lips, eyes, and eyebrows are frequently provided in a pen shaped tube, container, or applicator whereby said applicator is constructed with a cylindrical base portion and a cylindrical, tapering tip or end portion attached at one end to the base. An elongate, cylindrical stick of soft cosmetic material is attached to the base by a variety of means and extends through the tip or end portion until a portion thereof protrudes beyond an aperture formed within said tip or end portion. During use, the applicator is grasped primarily by the base and pointed, positioned, or otherwise manipulated so that the part of the cosmetic material extending beyond the aperture of the tip or end portion can make contact with a specific feature of the face (i.e. lips, eyes, eyebrows) in order to apply an appropriate quantity of cosmetic. Cosmetic applicators employ a variety of means to extend or retract the stick of cosmetic material in relation to the aperture of the tip or end portion. For instance, the stick of cosmetic material can slide within the base portion to extend beyond or retract within the aperture of the tip or end portion or the base portion can rotate while the tip or end portion remains stationary so that the stick of cosmetic material, rotating in a spiral fashion, extends from the base portion in a first direction beyond the aperture of the tip or end portion and retracts there from in a second direction upon reversing the rotation of the base portion.

While the designs and constructions of cosmetic applicators described earlier are adequate in most cases to apply appropriate quantities of soft cosmetic to various parts of the face, such applicators suffer from the disadvantage of not revealing the amount of cosmetic material remaining within the tip or end portion. As a typical tip or end portion is comprised of opaque material, usually solid plastic, the user cannot discern the amount of outstanding cosmetic material until said material is nearly depleted. The unexpected depletion of cosmetic material can pose a significant problem for the user should the material become used up during application if a replacement or alternate supply is unavailable. This situation is especially acute during a hurried or fast paced situation when the user has no chance of acquiring a replacement. Such an occurrence could be alleviated if the user was able to detect the amount of remaining cosmetic material so that the cosmetic applicator could be replaced well in advance of the cosmetic material's becoming depleted. One method of providing such detection is the provision of a cosmetic applicator constructed with a tip or end portion that is transparent or clear so that the stick of cosmetic material residing therein could be quickly evaluated as to the amount of remaining useful material.

### DISCUSSION OF THE PRIOR ART

Numerous designs for cosmetic applicators have been provided in the prior art. Even though these designs may be suitable for the specific individual purposes to which they address, they would not be suitable for the purposes of the present version of the invention. These designs are exemplified by U.S. Pat. No. 5,051,017, Clear Roll-On Bottle, issued to Yorks on Sep. 24, 1991 and U.S. Pat. No. 5,586,694, Perfume Applicator With Replaceable Cartridges, issued to Breidenbach et al. on Sep. 24, 1996.

As such, it may be appreciated that there is a continuing need for a new and improved cosmetic applicator with means to determine the amount or remaining cosmetic stick material. In these respects, the present version of the invention substantially departs from the conventional concepts and designs of the prior art, and in so doing provides an apparatus that substantially fulfills this need. Additionally, the prior patents and commercial techniques do not suggest the present inventive combination of component elements arranged and configured as disclosed herein.

The present invention achieves its intended purposes, objects, and advantages through a new, useful and unobvious combination of method steps and component elements, with the use of a minimum number of functioning parts, at a reasonable cost to manufacture, and by employing only readily available materials.

### SUMMARY

The present version of the invention, which will be described in greater detail hereinafter, relates to the field of cosmetic applicators. More specifically, this version of the invention is concerned with cosmetic applicators that are constructed with a clear or transparent tip or end portion that permits the user to determine the amount of cosmetic remaining within the applicator. My version of the invention overcomes all of the shortcomings listed previously, in addition to novel aspects that will be described in detail hereinafter.

Described briefly, according to a typical embodiment, the invention presents a cosmetic applicator with transparent tip that allows a user to discern the quantity of remaining cosmetic stick material. The applicator is constructed as a pen-shaped or cylindrical tube or container that is well known in prior art and design. One portion of the applicator is comprised of an opaque base portion constructed of plastic, wood, and the like. The base portion retains in sliding or rotating engagement a stick of cosmetic material that extends from or retracts within the base portion upon actuation of the sliding or rotating mechanism. A clear or transparent cylindrical tip is attached to the end of the base portion from which the stick of cosmetic material is extended or retracted. During use and non-use, the portion of the stick of cosmetic material surrounded by the transparent tip is clearly visible to the user. With this design therefore the user can quickly determine the quantity of remaining cosmetic material and purchase a replacement applicator well in advance of depleting the cosmetic material of the existing applicator.

My invention, therefore, resides not in any one of these features per se, but rather in the particular combination of all of them herein disclosed. It is distinguished from the prior art in this particular combination of all of its structures for the functions specified.

In as much as the foregoing has outlined rather broadly the more pertinent and important features of the present invention in order that the detailed description of the invention that follows may be better understood so that the present contribution to the art can be more fully appreciated. Additional features of the invention will be described hereinafter. It should be appreciated by those skilled in the art that the conception and the disclosed specific methods and structures may be readily utilized as a basis for modifying or designing other structures for carrying out the same purposes of the present invention. It should be realized by those skilled in the art that such equivalent methods and structures do not depart from the spirit and scope of the invention.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application nor is it intended to be limiting as to the scope of the invention in any way.

Accordingly, it is an object of my version of the invention to provide a low-cost, easy-to-manufacture, and easy-to-market cosmetic applicator with transparent tip.

A further object of my version of the invention is to provide an easy-to-use and versatile cosmetic applicator with transparent tip.

A significant object of the invention is to provide a cosmetic applicator with transparent tip that is constructed with a clear or transparent tip, said tip attached to the end of an applicator base portion from which a stick of cosmetic material is extended or retracted.

A final but very significant object of the invention is to provide a cosmetic applicator with transparent tip that permits a user to determine the amount of usable cosmetic material in order to acquire a replacement before the cosmetic material of the currently used applicator is used up.

For a better understanding of the invention, its operating advantages and the specific objects attained by its uses, reference should be made to the accompanying drawings and descriptive matter in which there is illustrated a preferred embodiment of the invention. The foregoing has outlined some of the more pertinent objects of the invention. These objects should be construed to be merely illustrative of some of the more prominent features and applications of the present invention. Many other beneficial results can be attained by applying the disclosed invention in a different manner or by modifying the invention within the scope of the disclosure. Accordingly, other objects and a fuller understanding of the invention may be had by referring to the summary of the invention and the detailed description of the preferred embodiment in addition to the scope of the invention illustrated by the accompanying drawings.

### BRIEF DESCRIPTION OF THE DRAWINGS

The foregoing and other objects, features and advantages of the invention will become more fully understood from the following description of the preferred embodiment of the invention as illustrated in the accompanying drawings in which like reference characters refer to the same parts throughout different views. The drawings are not necessarily to scale, emphasis instead being placed upon illustrating the principles of the invention.

FIG. 1 is a perspective view of a Cosmetic applicator with transparent tip in accordance with the present version of the invention.

### DRAWING REFERENCE NUMERALS

10 Cosmetic Applicator With Transparent Tip

12 Base

14 Tip

16 Cosmetic Stick

18 Upper End of Base

20 Lower End of Base

22 Rings

24 Rings

26 Upper End of Tip

28 Lower End of Tip

30 Aperture

### DESCRIPTION OF THE PREFERRED EMBODIMENT

### DESCRIPTION

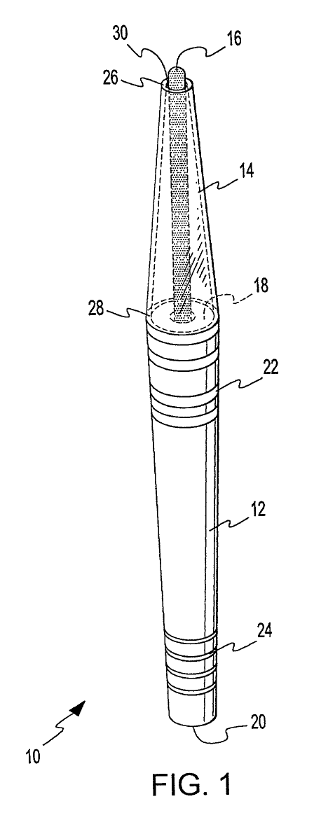

Referring now to the drawings and, in particular, to FIG. 1 wherein there is illustrated a typical embodiment of the cosmetic applicator with transparent tip 10. The present version of the invention 10 is fabricated primarily as a conventional pen-shaped cosmetic applicator having a cylindrical base 12 portion and a conically shaped tip 14 or end portion. A stick 16 of cosmetic material is secured to either a rotating or sliding mechanism within the base 12 in order to provide the means by which the stick 16 is extended from or retracted within the base 12 and tip 14. The mechanism comprises designs that are well known in prior art and conventional design and will not be discussed in detail within this disclosure as such designs are beyond the scope of the instant invention. In this disclosure, it is intended that the cosmetic applicator employ a rotating mechanism situated within the base 12 to extend or retract the stick 16 of cosmetic material, although other actuating mechanisms can be employed and remain within the spirit and scope of the invention. The base 12 is comprised of a first, upper end 18 with upper designating that portion closest to the point at which the stick 16 makes contact with the face of a user and a second lower end 20. The base 12 tapers from a wider diameter at the upper end 18 to a lesser diameter at the lower end 20. A first series of rings 22 protrude through the surface of the base 12 proximate to the upper end 18, and a second series of rings 24 protrude near the lower end 20. The rings 22, 24 can be used as an aid in grasping or turning the base 12 in relation to the tip 14 or to rotate any two sections of the base 12 with or without turning the tip 14 to extend or retract the stick 16 of cosmetic material. For instance, the base can be comprised of two sections separated by either the inner most ring of the first series of rings 22 or second series of rings 24. The base 12 is fabricated of lightweight, durable material that is economical to acquire and manufacture, such as wood, plastic, fiberglass, and the like and may be coated, painted, tinted, or dyed on the exterior with a variety of pigments, dyes, sealants, and various other surface treatments.

Referring again to FIG. 1, the tip 14 is attached to the base 12 either in stationary or rotating engagement depending upon the construction of the base 12 as referenced previously. The tip 14, which is fabricated from clear or transparent plastic or acrylic using various injection molding processes, is hollow and formed with a continuous sidewall that is bounded by a first circular end 26 of lesser diameter and a second circular end 28 of greater diameter at which said tip 14 is attached to the upper end 18 of the base 12. The tip 14 thus encloses a cavity into which the stick 16 of cosmetic material extends from the base 12 or recedes therefrom towards the base 12. Before use, the base 12 is rotated to extend the stick 16 through an aperture 30 formed at the upper end 26 of the tip 14 for a distance therefrom sufficient to easily apply an appropriate quantity of cosmetic material 16. After use, the base 12 is rotated in a reverse direction to draw the stick 16 within the cavity of the tip 14 in order to protect the stick 16 from damage during non-use and storage. As the stick 16 is used on a regular basis, it will diminish in length until it is no longer possible to extend it through the tip aperture 30. The transparent tip 14 will thus permit the user to detect the usable amount of cosmetic stick 16 material before said material 16 is depleted and to secure a replacement for the current cosmetic applicator 10 before such depletion occurs.

While this version of the invention has been illustrated and described in detail in the drawings and foregoing description, the same is to be considered as illustrative and not restrictive in character, it being understood that only the preferred embodiment has been shown and described and that all changes and modifications that come within the spirit of the version of the invention are desired to be protected. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

### CONCLUSION AND SCOPE OF INVENTION

From the foregoing, it will be understood by persons skilled in the art that an improved cosmetic applicator has been provided that incorporates a clear or transparent tip. The invention is relatively simple and easy to manufacture, yet affords a variety of uses. While my description contains many specificities, these should not be construed as limitations on the scope of the version of the invention, but rather as an exemplification of the preferred embodiment thereof. The foregoing is considered as illustrative only of the principles of the invention. Further, since numerous modifications and changes will readily occur to those skilled in the art, it is not desired to limit the invention to the exact construction and operation shown and described, and accordingly all suitable modifications and equivalents may be resorted to, falling within the scope of the invention. Although this invention has been described in its preferred form with a certain degree of particularity, it is understood that the present disclosure of the preferred form has been made only by way of example and numerous changes in the details of construction and combination and arrangement of parts may be resorted to without departing from the spirit and scope of the invention.

What is claimed is:

1. A cosmetic applicator for applying substantially solid cosmetic material comprising:
a container defining at least one cavity to receive the cosmetic material and including an aperture disposed adjacent a distal end portion thereof in communication with the at least one cavity, said container further including a transparent portion to determine the amount of the cosmetic material remaining within the cosmetic applicator.

2. The cosmetic applicator of claim 1 further including a cosmetic moving member for extracting and retracting the cosmetic material from and/or within the aperture.

3. The cosmetic applicator of claim 1 wherein at least a distal portion of the container is formed from transparent material.

4. A cosmetic applicator for applying substantially solid cosmetic material, comprising:
a base defining at least one cavity to receive at least a portion of the cosmetic material; and
a distal member connected to the base and defining a longitudinal cavity to receive at least a portion of the cosmetic material, the distal member including an aperture disposed adjacent a distal end portion thereof in communication with the longitudinal cavity;
wherein the distal member further including a transparent portion to determine the amount of the cosmetic material remaining within the cosmetic applicator.

5. The cosmetic applicator of claim 4 further including a cosmetic moving mechanism for extracting and retracting the cosmetic material from and/or within the aperture.

6. The cosmetic applicator of claim 5 wherein the cosmetic moving mechanism includes a cosmetic sliding mechanism disposed adjacent the base.

7. The cosmetic applicator of claim 5 wherein the cosmetic moving mechanism includes a spiral feed mechanism disposed adjacent the base.

8. The cosmetic applicator of claim 4 wherein the distal member is formed from transparent material to determine the amount of the cosmetic material remaining within the cosmetic applicator.

9. The cosmetic applicator of claim 4 wherein at least a distal portion of the distal member is formed from transparent material.

10. The cosmetic applicator of claim 4 wherein the cosmetic applicator has a pen-like shape.
